(19) United States
(12) Patent Application Publication
(10) Pub. No.: US 2025/0276159 A1
(43) Pub. Date: Sep. 4, 2025

(54) NASAL CATHETER ASSEMBLY AND METHODS OF USE

(71) Applicant: JStop, LLC, Bedminster, NJ (US)

(72) Inventor: Tanveer Janjua, Bedminster, NJ (US)

(73) Assignee: JStop, LLC, Bedminster, NJ (US)

(21) Appl. No.: 19/066,265

(22) Filed: Feb. 28, 2025

Related U.S. Application Data
(60) Provisional application No. 63/560,824, filed on Mar. 4, 2024.

(51) Int. Cl. A61M 25/10; A61M 25/00; A61M 39/24

(52) U.S. Cl. CPC A61M 25/1011; A61M 25/0043; A61M 25/0097; A61M 39/24; A61M 2025/006; A61M 2205/0216; A61M 2210/0618

(57) ABSTRACT

A nasal catheter assembly includes a balloon catheter that has a lumen and a first balloon in communication with the lumen. A hub is connected to the balloon catheter and defines an inlet port. The hub includes a housing that defines a plurality of ridges extending outwardly. The assembly also includes an absorbent member that has an outer surface configured to absorb a liquid and an inner surface that defines a cavity. A washer is moveably disposed over the hub housing and is disposed within the cavity of the absorbent member such that the washer engages the inner surface of the absorbent member. A nut is movably disposed over the hub housing and has at least one finger sequentially engageable with the plurality of ridges for incrementally moving and securing the washer and absorbent member at any one of a plurality of positions along the longitudinal axis of the housing.

### CROSS-REFERENCE TO RELATED APPLICATIONS

This application claims the benefit of the filing date of U.S. Provisional Application No. 63/560,824, filed Mar. 4, 2024, the disclosure of which is hereby incorporated herein by reference.

### BACKGROUND

Epistaxis (nosebleed) is a very common problem in the general public. Epistaxis can be divided into two types: anterior epistaxis (i.e., nosebleed originating at the front of the nasal cavity) and posterior epistaxis (i.e., nosebleed originating at the rear of the nasal cavity). Anterior epistaxis, which usually occurs at a collection of blood vessels at the nasal septum, is far more common than posterior epistaxis and can be more easily controlled because the blood vessels that usually cause this bleeding are small in size, superficially located, and easily compressible via internal nostril packing and/or external nose pinching. On the other hand, posterior epistaxis is more difficult to control because it is generally caused by a larger blood vessel (i.e., sphenopalatine artery) which is located in a region deep within the nasal cavity making it difficult to reach and compress via direct pressure.

In addition to being more difficult to control than anterior epistaxis, posterior epistaxis can pose serious health risks. Typically, patients who suffer from posterior epistaxis are elderly individuals who suffer from hypertension and may be on blood thinning medication which can significantly reduce the ability of blood to clot and bleeding to stop. Thus, these individuals are at high risk of excessive blood loss that can lead to heart attack, stroke, pneumonia, and even death. The goal is to stop the bleeding as soon as possible. This can typically be accomplished by an experienced Otolaryngologist (Ear, Nose and Throat Specialist). However, in many situations the only healthcare provider available may be an EMT (Emergency Medical Technician), paramedic, nurse, or an emergency room physician who may not have the training and experience to appropriately address posterior epistaxis with the currently available devices which are not user friendly and/or efficient. Therefore, further improvements are desirable.

### BRIEF SUMMARY OF THE DISCLOSURE

In one aspect of the present disclosure, a nasal catheter assembly includes a balloon catheter that has a first end, a second end, a lumen extending through the first end toward the second end, and a first balloon in communication with the lumen such that fluid flowing through the lumen fills and expands the first balloon. The assembly also includes a hub connected to the first end of the balloon catheter. The hub defines an inlet port in communication with the lumen of the balloon catheter such that fluid introduced through the inlet port is directed into the lumen. The hub includes a hub housing that defines a plurality of ridges that extend outwardly therefrom and transverse to a longitudinal axis of the hub. Also included in the assembly is an absorbent member that has an outer surface that is configured to absorb a liquid and an inner surface that defines a cavity. A washer is moveably disposed over the hub housing and is disposed within the cavity of the absorbent member such that the washer engages the inner surface of the absorbent member. Further, a nut is moveably disposed over the hub housing and has at least one finger sequentially engageable with the plurality of ridges for incrementally moving and securing the washer and absorbent member at any one of a plurality of positions along the longitudinal axis of the housing.

Additionally, the absorbent member may be a conical sponge that may have an opening that extends therethrough and which may communicate with the cavity. The balloon catheter may extend through the opening of the absorbent member. The inner surface of the absorbent member may taper inwardly from a first end toward a second end thereof, and the washer may include an outer surface that has a taper corresponding to the taper of the inner surface of the absorbent member such that advancing the washer within the cavity of the absorbent member applies an outward radial force on the absorbent member. The washer may include a convex surface, and the nut may include a concave surface engageable with the convex surface of the washer. The washer and hub may include corresponding flats which may permit slidable translation of the washer over the hub and may prohibit rotation of the washer relative to the hub. Also, at least a portion of the nut may be disposed within the cavity of the absorbent member and may engage the washer. At least one finger of the nut may be a flexible finger configured to permit axial translation of the nut in a first axial direction and resist axial translation in an opposite second axial direction when engaging any one of the ridges of the plurality of ridges. The plurality of ridges may be defined by at least one thread helically extending about the hub housing, and rotating the nut about the longitudinal axis of the hub housing in a first rotational direction may move the nut in the second axial direction.

Furthermore, the hub may include a first connector member and a second connector member which may be connected to the first connector member. The first connector member may define the inlet port and may include a one-way valve therein. The second connector member may define a connector end connected to the first end of the balloon catheter. The hub housing may include first and second housing portions disposed over at least a portion of each of the first and second connector members. The hub may further include a housing ring that may be disposed over at least a portion of the first and second housing portions and may secure the first and second housing portions together. The balloon catheter may further include a second balloon in communication with the lumen such that fluid flowing through the lumen may also fill and expand the second balloon.

In another aspect of the present disclosure, a nasal catheter assembly includes a balloon catheter that may have a first end, a second end, a lumen extending through the first end toward the second end, and a first balloon in communication with the lumen such that fluid flowing through the lumen fills and expands the first balloon. A hub is connected to the first end of the balloon catheter and defines an inlet port in communication with the lumen of the balloon catheter such that fluid introduced through the inlet port is directed into the lumen. The hub includes a hub housing that defines a plurality of ridges extending outwardly therefrom and transverse to a longitudinal axis of the hub. The assembly also includes an absorbent member that has a conically tapered outer surface that is configured to absorb a liquid and an inner surface that defines a cavity. A nut is movably disposed over the hub housing and is at least partially disposed within the cavity of the absorbent member. The nut has at least one finger that is sequentially engageable with the plurality of ridges for incrementally moving and securing the washer and absorbent member at any one of a plurality of positions along the longitudinal axis of the housing.

In a further aspect of the present disclosure, a nasal catheter assembly includes a balloon catheter that has a first catheter that defines a lumen, a second catheter disposed over the first catheter, and first and second balloons disposed over the first and second catheters. The first and second catheters each have a plurality of transverse openings that extend transverse to respective longitudinal axes thereof and are arranged relative to each other such that fluid flowing from the lumen and from the transverse openings fills and expands the first balloon and then the second balloon. A hub is connected to the balloon catheter and defines an inlet port in communication with the lumen of the balloon catheter such that fluid introduced through the inlet port is directed into the lumen. An absorbent member is slidably disposed over the hub.

Additionally, the assembly may also include a washer disposed within the absorbent member, which may be a conical sponge. The washer may be slidably disposed over the hub and rotationally constrained relative thereto. The first balloon may extend over an end of the first catheter, and the lumen of the first catheter may extend through the end. The second catheter may have an outer surface that may define a plurality of longitudinally extending ribs, and an inner surface that may define a plurality of longitudinally extending ribs. The ribs of the inner surface may engage the first catheter, and the ribs of the outer surface may interface with the first and second balloons. At least one pair of transverse openings of the first and second catheters that feed fluid to the first balloon may be aligned, and each of the transverse openings of the first and second catheters feeding fluid to the second balloon may non-aligned.

In yet another aspect of the present disclosure, a nasal catheter assembly includes a balloon catheter that has a pre-curve defining a center of curvature and first and second balloons. The first and second balloons are each configured to expand eccentrically away from the center of curvature when fluid is introduced to the first and second balloons through the balloon catheter. A hub is connected to the balloon catheter and defines a fluid inlet port in communication with the balloon catheter. Also, an absorbent member is slidably disposed over the hub.

Additionally, the balloon catheter may include a first catheter that may define a lumen of the balloon catheter and a second catheter that may be disposed over at least a portion of the first catheter. A line of adhesive may secure at least a portion of the second balloon to the second catheter. The first catheter may be made from a first material, the second catheter may be made from a second material, and the second material may be more flexible than the first material. The first material may be polyetheretherketone, and the second material may be silicone.

### BRIEF DESCRIPTION OF THE DRAWINGS

FIG. 1 is a schematic view of an exemplary embodiment of a nasal catheter system.

FIG. 2 is an exploded view of an exemplary embodiment of a nasal catheter assembly of the system of FIG. 1.

FIG. 3A is an elevational view of an exemplary embodiment of a balloon catheter of the nasal catheter assembly of FIG. 2.

FIG. 3B is a cross-sectional view of the balloon catheter taken along a line B-B of FIG. 3A.

FIG. 3C is an elevational view of the balloon catheter of FIG. 3A in a pre-curved configuration.

FIG. 4A is an elevational view of an exemplary embodiment of a stylet of the balloon catheter of FIG. 3A.

FIG. 4B is an elevational view of the stylet of FIG. 4A rotated 90 degrees relative to FIG. 4A.

FIG. 4C is an end view of the stylet of FIG. 4A.

FIG. 5A is an elevational view of an exemplary embodiment of a backbone of the balloon catheter of FIG. 3A.

FIG. 5B is an elevational view of the backbone of FIG. 5A rotated 90 degrees relative to FIG. 5A.

FIG. 5C is an end view of the backbone of FIG. 5A.

FIG. 6 is an elevational view an exemplary embodiment of a hub of the nasal catheter assembly of FIG. 2.

FIG. 7 is an elevational view of an exemplary embodiment of a first connector member of the hub of FIG. 6.

FIG. 8 is an elevational view of an exemplary embodiment of a second connector member of the hub of FIG. 6.

FIG. 9 is a perspective view of an assembly that includes the balloon catheter of FIG. 5A, first connector member of FIG. 7, and second connector member of FIG. 8.

FIG. 10A is a perspective view of an exemplary embodiment of a hub housing of the hub of FIG. 6.

FIG. 10B is an end view of the hub housing of FIG. 10A.

FIG. 10C is a perspective view of a first housing portion of the hub housing of FIG. 10A.

FIG. 10D is an elevational view of the first housing portion of FIG. 10C.

FIG. 11A is a perspective view of an exemplary embodiment of a housing ring of the hub of FIG. 6.

FIG. 11B is a cross-sectional view of the housing ring of FIG. 11A taken along a midline thereof.

FIG. 12 is a perspective view of an exemplary embodiment of an absorbent member of the nasal catheter assembly of FIG. 2.

FIG. 13A is a perspective view of an exemplary embodiment of a washer of the nasal catheter assembly of FIG. 2.

FIG. 13B is an elevational view of the washer of FIG. 13A.

FIG. 14 is a cutaway view of an assembly that includes the absorbent member of FIG. 12 and the washer of FIG. 13A.

FIG. 15A is a perspective view of an exemplary embodiment of a nut of the nasal catheter assembly of FIG. 2.

FIG. 15B is a cross-sectional view of the nut of FIG. 15A taken along a midline thereof.

FIGS. 16A-16D illustrate an exemplary method of using the nasal catheter assembly.

### DETAILED DESCRIPTION

When referring to specific directions in the following discussion of exemplary devices, systems, and assemblies, it should be understood that such directions may be described with regard to the orientation and position of such devices, systems, and assemblies relative to the human body. Thus, the term “anterior” refers to a location closer to the front of the body or the face, and the term “posterior” refers to a location closer to the back of the body. Reference to specific directions may also be made relative to the operator of the exemplary devices, systems, and assemblies disclosed herein. In this regard, the term “proximal” means closer to the operator or in a direction toward the operator, and the term “distal” means more distant from the operator or in a direction away from the operator. Also, it should be understood, as used herein, the term “posterior” is interchangeable with the term “distal,” and the term “anterior” is interchangeable with the term “proximal.”

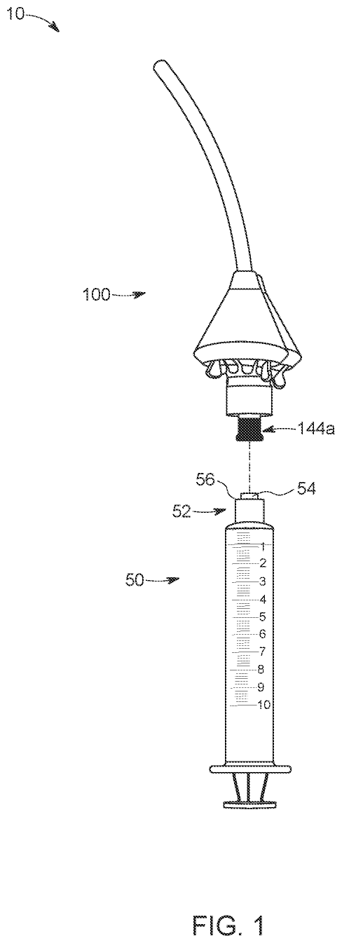

FIG. 1 depicts a nasal catheter system 10 according to an embodiment of the present disclosure. System 10 generally includes a fluid delivery device 50 and a nasal catheter assembly 100. Fluid delivery device 50 may be a syringe with a connector 52 for connecting to nasal catheter assembly 100. For example, connector 52 of fluid delivery device 50 may be a Luer lock connector which may include a male taper member 54 and a threaded collar 56. However, other connectors are contemplated, such as a Luer slip connector, snap-fit connectors, barbed connectors, and the like, for example. Fluid delivery device 50 may be configured to transfer fluid to nasal catheter assembly 100 and preferably in metered volumes. Such fluid may include liquids (e.g., saline) and/or gases (e.g., air).

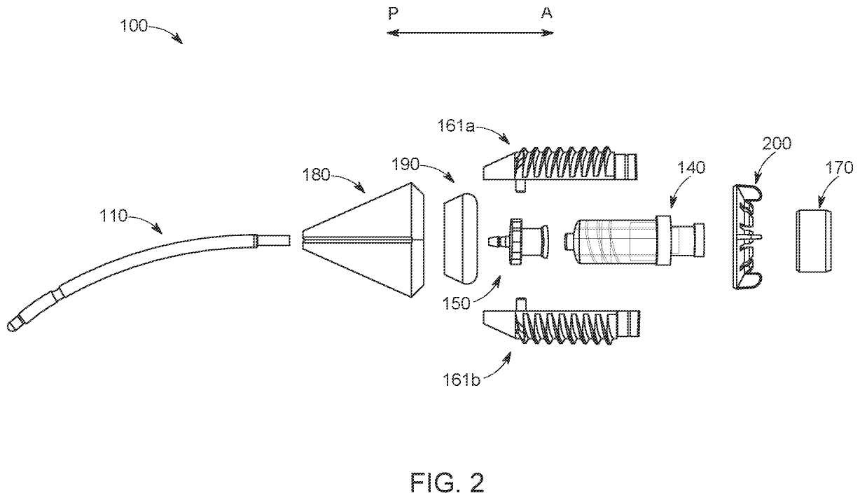

FIGS. 2-15B depict nasal catheter assembly 100. Nasal catheter assembly 100 generally includes a balloon catheter 110, a hub 102, an absorbent member 180, a washer 190, and a nut 200.

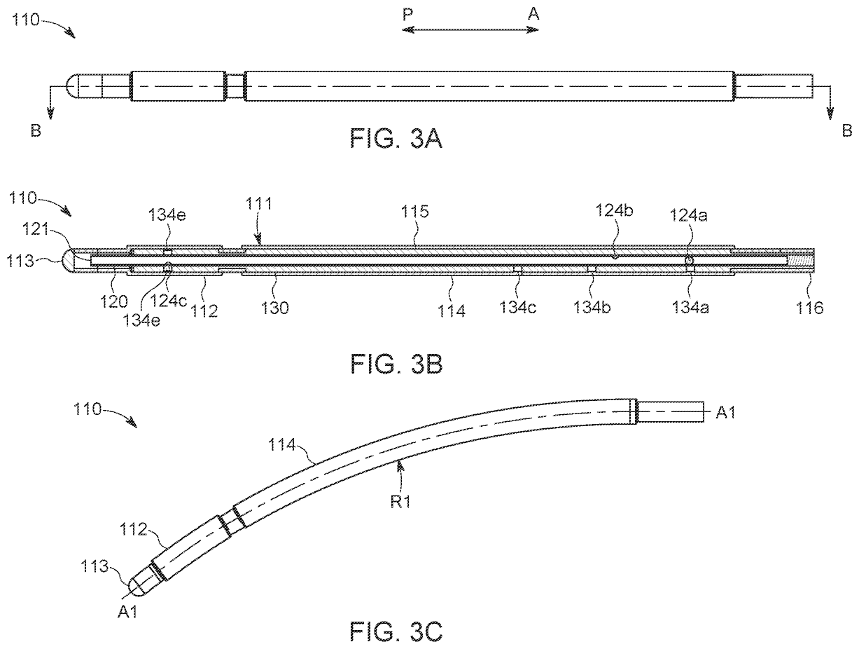

FIGS. 3A-5C depict balloon catheter 110. Balloon catheter 110 preferably includes two balloons. However, in some embodiments, a single balloon may be provided. In the particular embodiment depicted, balloon catheter 110 includes a first balloon or posterior balloon 112 and a second balloon or anterior balloon 114. More specifically, balloon catheter 110 may include a stylet 120, a backbone 130, and a balloon skin 111. Posterior balloon 112 may be of a same or different size than anterior balloon 114. In some examples, posterior balloon 112 may be smaller than anterior balloon 114. Posterior balloon 112 may be axially spaced from anterior balloon 114.

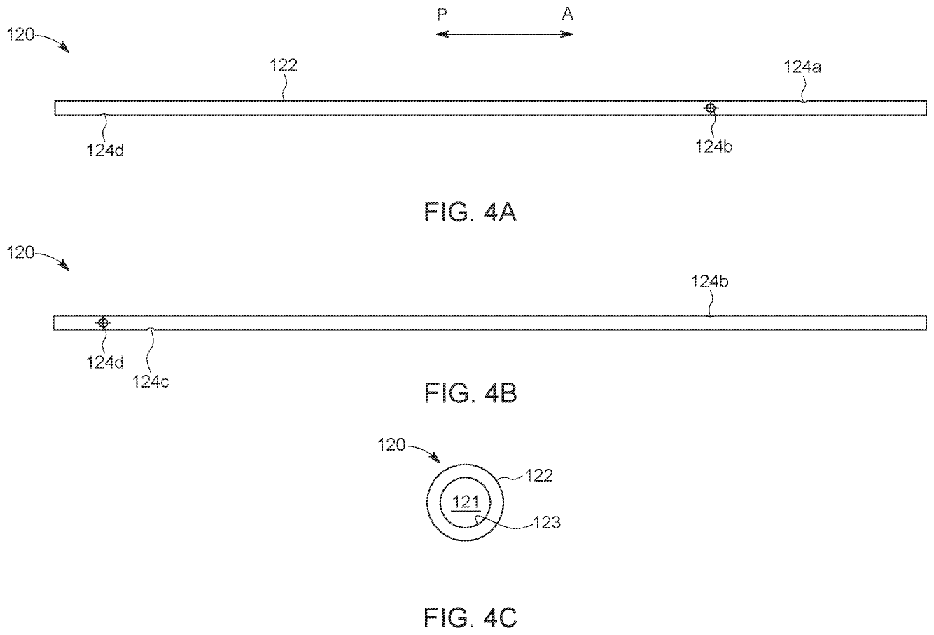

FIGS. 4A-4C depict stylet 120. Stylet or first catheter 120 may be an extrusion of rigid polymer material, such as polyetheretherketone (“PEEK”), for example. Stylet 120 may include an outer surface 122 and an inner surface 123 which both may be cylindrical, as shown in FIG. 4C. Inner surface 123 may define a lumen 121 which may extend along and through the entire length of stylet 120. Such lumen 121 may also be the lumen for balloon catheter 110 and, as such, may be the main channel delivering fluid to balloons 112 and 114. Stylet 120 may be divided between a posterior portion and an anterior portion. As shown in FIG. 4A, the double-headed arrow illustrates the posterior (“P”) and anterior (“A”) directions. Stylet 120 may include a plurality of openings 124 which may extend through a wall of stylet 120 in a direction transverse to a longitudinal axis of stylet 120. As shown in FIGS. 4A and 4B, the anterior portion of stylet 120 may include a first opening 124a and second opening 124b, and the posterior portion of stylet 120 may include a third opening 124c and a fourth opening 124d. In the embodiment depicted, each of these openings 124a-d are blind openings in that they extend through the stylet wall and intersect lumen 121, but do not continue further back through the wall. Additionally, openings 124a-d are not only offset longitudinally, but may also be offset rotationally. In other words, first opening 124a may be rotationally offset 90 degrees relative to second opening 124b, and third opening 124c may be rotationally offset 90 degrees relative to fourth opening 124d. Furthermore, first and fourth openings 124a, 124d may be offset 180 degrees, and second and third openings 124b, 124c may be offset 180 degrees. Thus, none of the openings 124a-d of stylet 120 may lie on the same side of stylet 120. Thus, fluid flowing through lumen 121 of stylet 120 not only travels axially through lumen 121 but may also travel transversely in four orthogonal directions through openings 124a-d. Although four openings 124a-d are shown, more or less openings are contemplated which may depend on the desired volumetric flow through each portion of stylet 120 and also the length of the stylet 120.

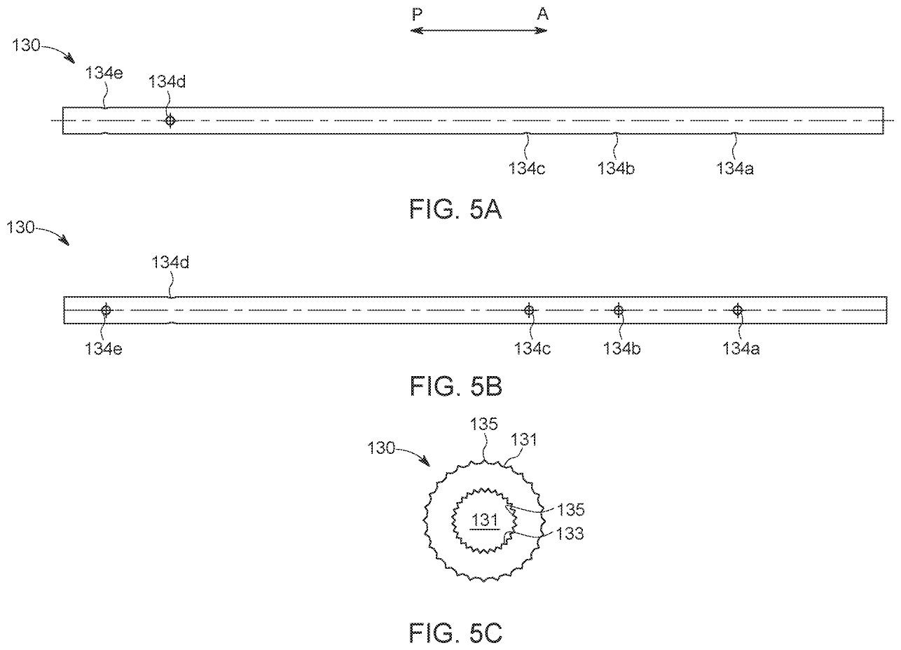

FIGS. 5A-5C depict backbone 130. Backbone or second catheter 130 may be an extrusion of flexible polymer material, such as silicone, for example. Backbone 130 may include an outer surface 131 and an inner surface 133 which both may be cylindrical, as shown in FIG. 5C. However, unlike stylet 120, outer surface 131 and inner surface 133 may each include a plurality of longitudinally extending ribs 135 arrayed about a longitudinal axis of backbone 130. Such ribs 135 may define longitudinal channels which may direct fluid axially toward opposing ends of backbone 130. Inner surface 133 may define a lumen 131 which may extend along and through the entire length of backbone 130. Such lumen 131 may be configured to receive stylet 120. Backbone 130 may include a plurality of openings 134 which extend through a wall of backbone 130 in a direction transverse to a longitudinal axis of backbone 130. As shown in FIGS. 5A and 5B, the anterior portion of backbone 130 may include a first opening 134a, a second opening 134b, and a third opening 134c, and the posterior portion of backbone 130 may include a fourth opening 134d and a fifth opening 134c. In the embodiment depicted, first, second, and third openings 134a-c may be blind openings and may each lie in a single plane and extend through the same side of backbone 130. On the other hand, fourth and fifth openings 134d, 134c may be through openings in that they may extend through the wall of backbone 130, intersect lumen 131, and continue extending back through the wall. Fourth and fifth openings 134d, 134e may be rotationally offset by 90 degrees. However, as shown, fifth opening 134e may be coplanar with openings 134a-c. Like stylet 120, more or less openings 134 are contemplated at least depending on the desired volumetric flow and the length of balloon catheter 110.

As best shown in FIG. 3B, a posterior end of stylet 120 extends from a posterior end of backbone 130, and balloon skin 111 extends over both backbone 130 and stylet 120. Balloon skin 111 is segmented between first balloon 112 and the second balloon 114. First balloon 112, which also defines a tip 113 of balloon catheter 110, extends over the posterior end of stylet 120 and backbone 130 including third and fourth openings 124c-d of stylet 120 and fourth and fifth openings 134d, 134c of backbone 130. As shown, third opening 124c of stylet 120 is aligned with fifth opening 134e of backbone 130. Thus, fluid exiting these openings 124c, 134e will tend to expand first balloon 112 eccentrically relative to a longitudinal axis A1 of balloon catheter 110.

The second balloon 114 extends over the anterior portions of stylet 120 and backbone 130 including first and second openings 124a-b of stylet 120 and first, second, and third openings 134a-c of backbone 130. However, unlike with first balloon 112, none of the openings 124a-b, 134a-c between stylet 120 and backbone 130 here are aligned. Thus, fluid traveling through first and second openings 124a-b of stylet 120 meets more resistance than at the posterior portion of balloon catheter 110 and consequently will take longer to exit into second balloon 114 from openings 134a-c which has a resulting effect of differential inflation of first and second balloons 112, 114 from a single, main lumen 121. Such differential inflation may be further facilitated by the posterior end of lumen 121 dispensing directly into first balloon 112, as best shown in FIG. 3B which, as mentioned above, is in contrast to the indirect route from stylet 120, along ribs 135, and through backbone 130 for filling second balloon 114. Differential thicknesses between balloons 112 and 114 may also be utilized to achieve the desired differential filling. In this regard, the material thickness of first balloon 112 may be thinner than the material thickness of second balloon 114. Thus, in some embodiments any or all of such features may be utilized in balloon catheter 110 so that the fill rate of first balloon 112 is generally greater than that of second balloon 114 such that first balloon 112 expands faster than second balloon 114 and reaches a fully expanded/filled state prior to second balloon 114. In such embodiments, second balloon 114 may at some point expand while first balloon 112 is also expanding but at a slower rate. However, in other embodiments, the resistance to filling second balloon 114 may be tailored such that the pressure needed to expand second balloon 114 may only be achieved after first balloon 112 has been expanded to the requisite extent to apply the appropriate pressure to the tissues within the nasal cavity. Thus, in such other embodiments, second balloon 114 may not begin to expand until first balloon 112 is fully expanded at the target location within the nasal cavity. In any event, the differential filling of balloons 112 and 114 results in first balloon 112 being lodged first within the nasal cavity at a target location to treat posterior epistaxis. Moreover, differential filling from a single, main lumen 121 and a single interface 144a with fluid delivery device 50 simplifies the procedure particularly for medical personnel inexperienced in treating posterior epistaxis.

Additionally, at least because the openings 134a-c are each aligned at the same side of backbone 130, once fluid exits such openings 134a-c, second balloon 114 will tend to expand eccentrically in the same eccentric direction as first balloon 112. To further facilitate eccentric expansion, a line of adhesive 115 may be applied between backbone 130 and first and/or second balloons 112, 114 at a side of catheter 110 opposite the eccentric expansion which has the effect of preventing expansion at the side of adhesive 115. Although first and second balloons 112, 114 are described as being inflatable eccentrically, balloon catheter 110 may be configured such that first and second balloons 112, 114 expand symmetrically and radially outwardly relative to stylet 120 and backbone 130.

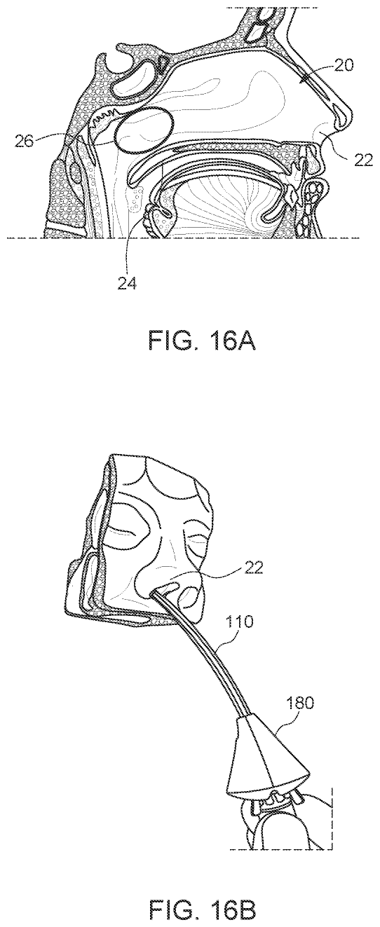

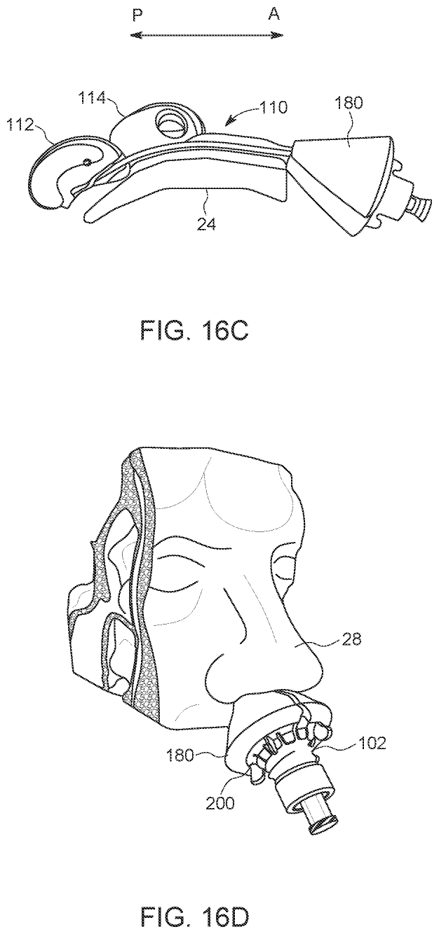

Also, as shown in FIG. 3C, balloon catheter 110 may be provided with a pre-curve which may be facilitated by the generally rigid stylet 120. Such pre-curve may define a center of curvature and a radius of curvature RI such that balloon catheter 110 has a concave curvature generally matching a convex curvature of a patient's palate anatomy (see FIG. 16A). The rigid structure of balloon catheter 110 coupled with the pre-curve may help insertion of balloon catheter 110 into a patient's nasal cavity. In addition to the pre-curve, the eccentric expansion mentioned above may be configured such that first and second balloons 112, 114 expand outwardly in a direction away from the center of curvature. This may allow balloon catheter 110 to rest in the desired position on the patient's palate while the balloons 112, 114 expand outwardly into engagement with the nasal cavity.

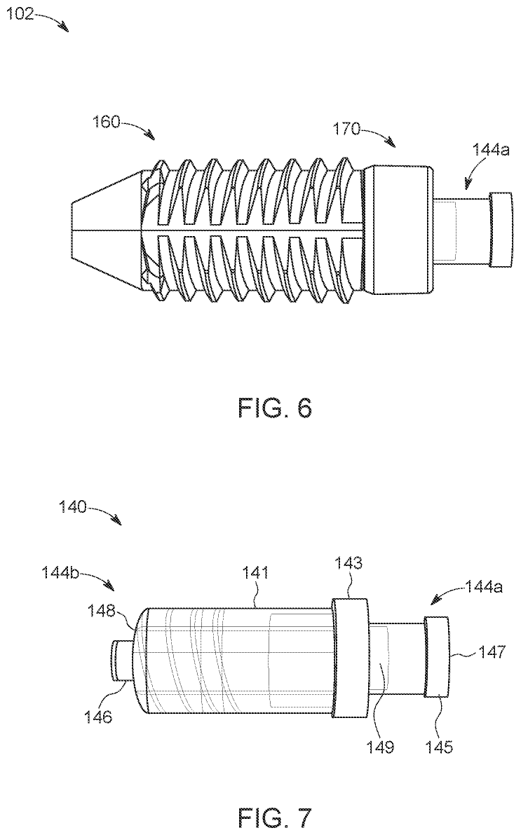

FIGS. 6-11B depict hub 102 of nasal catheter assembly 100. In the depicted embodiment, hub 102 generally includes a first connector 140, a second connector 150, a hub housing 160, and a housing ring 170.

FIG. 7 depicts first connector 140. First connector 140 generally includes a connector body 141, a first connector end 144a, and a second connector end 144b. Connector body 141 may be cylindrical and may include a flange 143 extend radially outwardly therefrom at an anterior end thereof. A valve 149 may be disposed within body 141. Such valve 149 may be a one-way check valve permitting flow from first connector end 144a to second connector end 144b, but not vice versa. First connector end 144a may define an inlet port 147 for fluid introduction into first connector 140 and ultimately into lumen 121 of balloon catheter 110. First connector end 144a may be configured to connect to fluid delivery device 50. In this regard, first connector end 144a may include a Luer lock connector which may include a female taper opening 147 and a flange 145. However, other connectors are contemplated so as to correspond with fluid delivery device 50, such as a Luer taper and the like. Second connector end 144b may be configured to connect to second connector 150 and, in this regard, may include Luer lock connector which may include a male taper member 146 and threaded collar 148, as shown. Again, other connectors are contemplated.

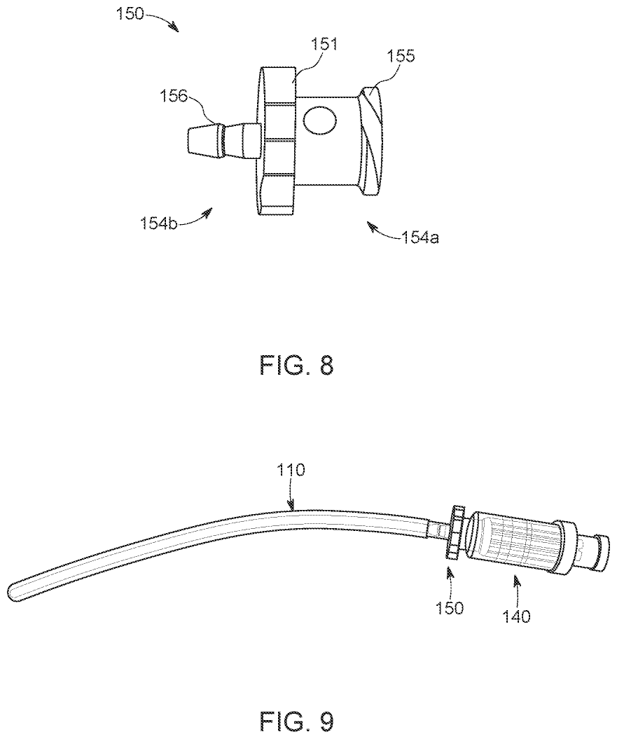

FIG. 8 depicts second connector 150 which may include a body 151, a first connector end 154a, and a second connector end 154b. Second connector 150 may operate as an adapter/coupling between first connector 140 and balloon catheter 110. In this regard, first connector end 154a may be configured to connect to second connector end 144b of first connector 140 and, as such, may include a Luer lock. On the other hand, second connector end 154b may include a barbed taper 156 for a fluid-tight engagement between second connector 156 end and balloon catheter 110.

FIG. 9 depicts an assembly of first and second connectors 140, 150 and balloon catheter 110. In this regard, second connector end 144b of first connector 140 is connected to first connector end 154a of second connector 150. Also, second connector end 154b of second connector 150 is connected to anterior end of balloon catheter 110 such that lumen 121 is in communication with inlet port 147 of first connector 140.

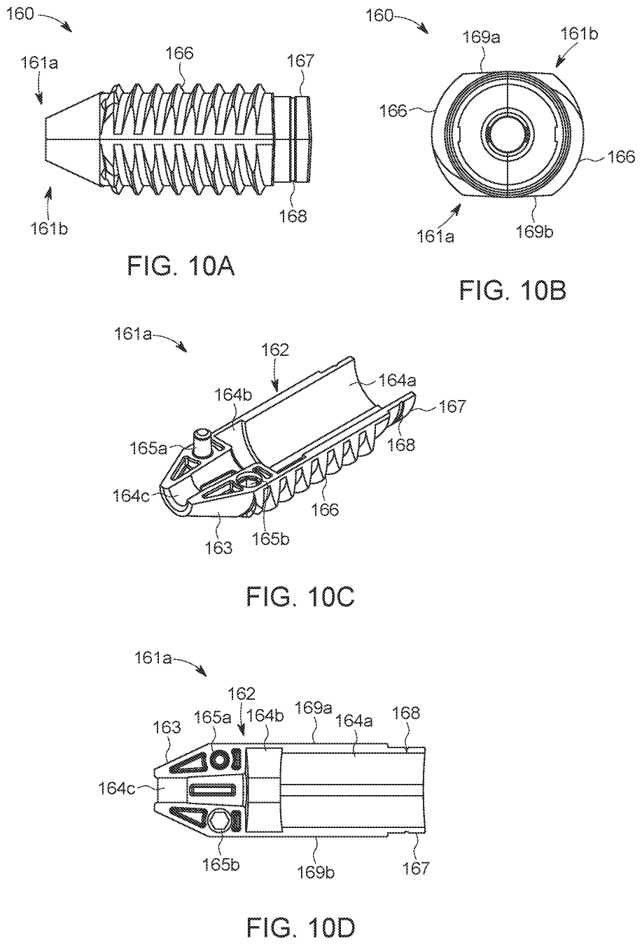

FIGS. 10A-10D depict the hub housing 160. Hub housing 160 may include a first housing portion 161a and a second housing portion 161b which may connect together over first and second connectors 140, 150 to form hub 102. First housing portion 161a is shown in FIGS. 10C and 10D and is like second housing portion 161b. First housing portion 161a includes a body 162, a nose cone 163 extending from one end of body 162, and a neck 167 extending from another end of body 162. Nose cone 163 may be conically tapered and may include a post 165a and a post hole 165b for corresponding engagement with the same features of second housing portion 161b. Body 162 may be cylindrical and may include a plurality of ridges 166 arrayed along a longitudinal axis thereof and extending transverse relative to the longitudinal axis. Such ridges 166 may be in the form of one or more helical threads. Alternatively, such ridges 166 may be in the form of ratchet teeth. Neck 167 may not include ridges 166 but may include a groove 168. As mentioned, first housing portion 161a may be positioned over first and second connectors 140, 150 and may thereby form a shell or housing thereof. In this regard, first housing portion 161a may include a first surface 164a configured to receive body 141 of first connector 140, a second surface 164b configured to receive body 151 of second connector 150, and a third surface 164c configured to receive an anterior portion of balloon catheter 110. As such, each of these surfaces 164a-c may be partially cylindrical and, when first and second housing portions 161a-b come together, form corresponding cavities or openings for such features. Additionally, when first and second housing portions 161a-b are connected together, as shown in FIGS. 10A and 10B, threads 166 may be interrupted adjacent to an interface between housing portions 161a-b and opposing flats 169a-b may be formed which extend longitudinally along such interface, as best shown in FIGS. 10A and 10B.

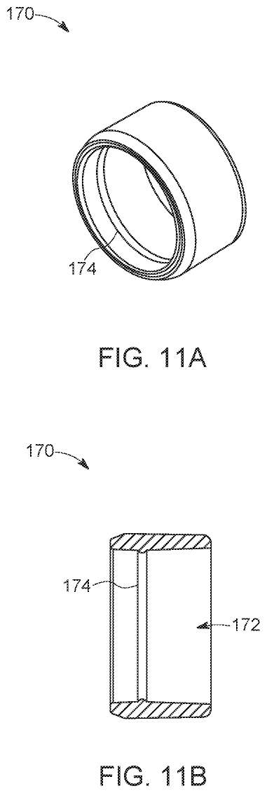

FIGS. 11A and 11B depict the housing ring 170. Housing ring 170 may be a ring or annulus which defines an opening 172 extending therethrough which may be configured to receive an end portion of hub 102, which is formed by the necks 167 of first and second housing portions 161a-b. Opening 172 may also be configured to receive flange 143 of first connector 140. Ring 170 may also include a circumferential protrusion 174 extending radially inwardly. Such protrusion 174 may engage groove 168 of hub housing 160 in a snap-fit manner. Such connection between housing ring 170 and hub housing 160 secures first and second housing portions 161a-b together.

Although hub 102 has been described herein as including a hub housing 160, first and second connectors 140, 150, and housing ring 170, it should be understood that in some embodiments, first and second connectors 140, 150 may be integral such that they form a single connector with the desired connector ends (e.g., ends 144a, 154b). In other embodiments, all of the aforementioned components of hub 102 may be integral such that hub 102 forms a single monolithic component that includes connector ends (e.g., ends 144a, 154b) and ridges 166 on its exterior.

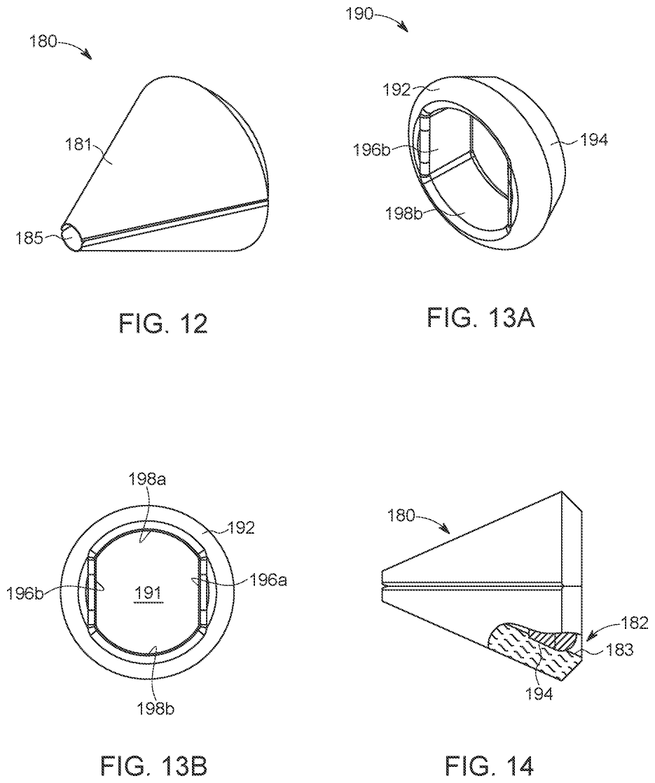

FIG. 12 depicts an absorbent member 180 of the nasal catheter assembly 100. In the embodiment depicted, absorbent member 180 is a conical sponge that has a conically tapered outer surface 181 configured to absorb liquids, such as blood. In some embodiments, outer surface 181 may also include a coating or a covering. For example, outer surface 181 may include a Telfa® lining or covering which resists sticking to blood and nasal mucosa thereby making removal of absorbent member 180 from the patient easier than without the Telfa® lining. In another example, outer surface 181 may include a tranexamic acid (“TXA”) coagulant coating which can help stop bleeding. Absorbent member 180 also includes an inner surface 183 that defines a cavity 182. Such inner surface 183 may be tapered inwardly from an anterior end to a posterior end thereof, as best shown in FIG. 14. As such, cavity 182 may be conically tapered. Cavity 182 may extend through the posterior end of absorbent member 180 so as to form an opening 185 configured to slidably receive balloon catheter 110. Although absorbent member 180 is described as being a sponge, absorbent member 180 can be made from other absorbent material, such as gauze, for example. However, a sponge material is preferable as it highly absorbent, is soft, and generally retains its shape.

FIGS. 13A and 13B depict a washer 190 of the nasal catheter assembly 100. Washer 190 is generally annular such that it has an opening 191 extending therethrough. In the embodiment depicted, such opening 191 is defined by first and second curved surfaces 198a-b and first and second flat surfaces 196a-b, as best shown in FIG. 13B. First and second curved surfaces 198a-b are disposed opposite each other and are concavely curved. Such curved surfaces 198a-b are configured to be positioned over threads 166 of hub 160 and to slide along such threads 166. In other words, a diameter defined between inner curved surfaces 198a-b is greater than a major diameter of the threads 166 of hub 160. Flat surfaces 198a-b are disposed opposite each other and are configured to be positioned along flats 169a-b of hub 160. Thus, when washer 190 is positioned over hub 160, washer 190 may be axially translatable along hub 160, but may be prohibited from rotation relative to hub 160 at least due to the arrangement of flats 169a-b. Washer 190 may also include a tapered outer surface 194 and a curved outer surface 192. Tapered outer surface 194 may be a conically tapered surface which may have a taper corresponding to the taper of inner surface 183 of absorbent member 180. In this regard, washer 190 may be positioned within cavity 192 of absorbent member 180 such that tapered outer surface 181 engages tapered inner surface 183 which may have the effect of washer 190 pushing radially outwardly on absorbent member 180. This may provide support for absorbent member 180, particularly when driven into a nostril of a patient. Curved outer surface 192 may be convexly curved, may intersect tapered surface 194 and may form at least a portion of an anterior side of washer 190.

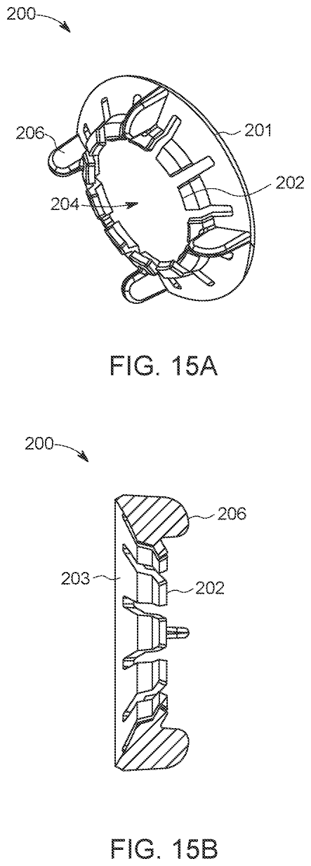

FIGS. 15A and 15B depict a nut 200 of nasal catheter assembly 100. Nut 200 may be a push nut. In this regard, nut 200 may include an annular body 201 defining an opening 204 extending therethrough. Annular body 201 may include a plurality of fingers or flexible members 202 extending radially inwardly into opening 204. Such fingers 202 may flex in one direction while being rigid in another direction. As such, when fingers 202 engage a ridge 166 of hub 160, pushing nut 200 in a first direction may cause fingers 202 to flex and nut 200 to advance to next ridge 166. Conversely, pushing nut 200 in a second direction opposite the first direction may be resisted by fingers 202 engaging a ridge 166. Thus, fingers 202 and ridges 166 may act like a ratchet which can be reversed by rotating nut 200 in a first rotational direction. Also, rather than pushing nut 200 along ridges 202, nut 200 may be advanced by rotating nut 200 in a second rotational direction. Nut 200 may also include a plurality of tabs or wings 206 which may extend anteriorly from annular body 201 and may facilitate rotation of nut 200. As shown in FIG. 15B, nut 200 may further include a concavely curved posterior surface 203. The concave curvature of surface 203 may correspond to a convex curvature of curved surface 192 of washer 190. In this regard, convex and concave surfaces 190, 203 may engage each other to provide uniform axial force distribution during axial translation while facilitating rotation of nut 200 relative to washer 190.

Although assembly 100 is depicted and described above as including washer 190, some embodiments of assembly 100 may not include washer 190. In this regard, annular body of nut 200 may be positioned at least partially within cavity 182 of absorbent member 180 (see e.g., FIG. 16D) such that absorbent member 180 is moveable along hub 160 in an anterior-posterior direction as previously described. As such, nut 200 facilitates the translation of absorbent member 180 and its positioning within a nostril of a patient.

FIGS. 16A-16D depict a method of using nasal catheter assembly 100. As illustrated in FIG. 16A, posterior epistaxis typically originates at a target location 26 within a posterior region of the nasal cavity 20. Also, as illustrated in FIG. 16A, the path for reaching the target location 26 is generally curved along the palate 24 (hard and soft) from a designated nostril 22. The pre-curve of balloon catheter 110 helps conform to this natural curvature such that those with less experience treating posterior epistaxis may find it easier to guide balloon catheter 110 to the target location 26 within the nasal cavity 20 as compared to catheters without a pre-curve. In this regard, balloon catheter 110 may be inserted into a designated nostril 22 with the concave curve of balloon catheter 110 pointing downward or inferiorly so that the concave curve matches the convex curve of the palate 24, as illustrated in FIGS. 16B and 16C.

Once the posterior end of balloon catheter 110 is properly positioned at the target location 26, fluid delivery device 50 may be used to deliver fluid through inflation port 147 of hub 102. Due to the configuration of balloon catheter 110, as described above, first balloon 112 inflates first thereby providing direct pressure against the target location 26. First balloon 112 also lodges into the nasopharynx and seals it completely hence stopping any blood from entering the throat. As fluid continues to be delivered to nasal catheter assembly 100 from a single inlet port 147, the fluid eventually makes its way into second balloon 114 such that it is expanded in the anterior portion of the nasal cavity 20, as illustrated schematically in FIG. 16C. The eccentric expansion of first and second balloons 112, 114 allows catheter 110 to rest on the palate 24 which helps maintain its position relative to the target location 26 as each balloon 112, 114 is inflated.

Once the balloons 112, 114 are sufficiently inflated, absorbent member 160 is driven toward the designated nostril 22. This may be done by pushing nut 200 axially toward the external nose 28 which slides absorbent portion 180 into the designated nostril 22. The fingers 202 of nut 200 and ridges 166 of hub 160 prevent the absorbent portion 180 from advancing out of the nostril 22. Additionally, axial pressure imposed on washer 190 by nut 200 creates corresponding radial and axial pressure from within absorbent member 180 which helps seal the nostril 22 from any leakage and further helps apply pressure from within the nostril on Kiesselbach's plexus, which is the anatomical feature responsible for anterior epistaxis. Thus, assembly 100 can address both posterior and anterior epistaxis, concurrently. When it comes time to remove the assembly 100 from the patient's nasal cavity 20, nut 200 may be rotated in a first direction which backs up nut 200 allowing washer 190 and absorbent portion to back out of the nostril 22. Balloon catheter 110 may be cut or otherwise drained to deflate balloons 112, 114 and remove catheter 110.

Although the subject matter disclosed herein has been described with reference to particular embodiments, it is to be understood that these embodiments are merely illustrative of the principles and applications of the present invention. It is therefore to be understood that numerous modifications may be made to the exemplary embodiments and that other arrangements may be devised without departing from the spirit and scope of the present disclosure as defined by the appended claims.

1. A nasal catheter assembly, comprising:
a balloon catheter having a first end, a second end, a lumen extending through the first end toward the second end, and a first balloon in communication with the lumen such that fluid flowing through the lumen fills and expands the first balloon;
a hub connected to the first end of the balloon catheter and defining an inlet port in communication with the lumen of the balloon catheter such that fluid introduced through the inlet port is directed into the lumen, the hub having a hub housing defining a plurality of ridges extending outwardly therefrom and transverse to a longitudinal axis of the hub;
an absorbent member having an outer surface configured to absorb a liquid and an inner surface defining a cavity;
a washer moveably disposed over the hub housing and being disposed within the cavity of the absorbent member such that the washer engages the inner surface of the absorbent member; and
a nut movably disposed over the hub housing and having at least one finger sequentially engageable with the plurality of ridges for incrementally moving and securing the washer and absorbent member at any one of a plurality of positions along the longitudinal axis of the housing.

2. The assembly of claim 1, wherein the absorbent member is a conical sponge having an opening extending therethrough and in communication with the cavity, the balloon catheter extending through the opening of the conical sponge.

3. The assembly of claim 1, wherein the inner surface of the absorbent member tapers inwardly from a first end toward a second end thereof, and the washer includes an outer surface that has a taper corresponding to the taper of the inner surface of the absorbent member such that advancing the washer within the cavity of the absorbent member applies an outward radial force on the absorbent member.

4. The assembly of claim 1, wherein the washer includes a convex surface, and the nut includes a concave surface engageable with the convex surface of the washer.

5. The assembly of claim 1, wherein the washer and hub include corresponding flats which permit slidable translation of the washer over the hub and prohibit rotation of the washer relative to the hub.

6. The assembly of claim 1, wherein at least a portion of the nut is disposed within the cavity of the absorbent member and engages the washer.

7. The assembly of claim 1, wherein the at least one finger of the nut is a flexible finger configured to permit axial translation of the nut in a first axial direction and resist axial translation in an opposite second axial direction when engaging any one of the ridges of the plurality of ridges.

8. The assembly of claim 7, wherein the plurality of ridges are defined by at least one thread helically extending about the hub housing, and rotating the nut about the longitudinal axis of the hub housing in a first rotational direction moves the nut in the second axial direction.

9. The assembly of claim 1, wherein the hub further includes a first connector member and a second connector member connected to the first connector member, the first connector member defining the inlet port and including a one-way valve therein, the second connector member defining a connector end connected to the first end of the balloon catheter.

10. The assembly of claim 9, wherein the hub housing includes first and second housing portions disposed over at least a portion of each of the first and second connector members.

11. The assembly of claim 10, wherein the hub further includes a housing ring disposed over at least a portion of the first and second housing portions and securing the first and second housing portions together.

12. The assembly of claim 1, wherein the balloon catheter further includes a second balloon in communication with the lumen such that fluid flowing through the lumen also fills and expands the second balloon.

13. A nasal catheter assembly, comprising:
a balloon catheter having a first end, a second end, a lumen extending through the first end toward the second end, and a first balloon in communication with the lumen such that fluid flowing through the lumen fills and expands the first balloon;
a hub connected to the first end of the balloon catheter and defining an inlet port in communication with the lumen of the balloon catheter such that fluid introduced through the inlet port is directed into the lumen, the hub having a hub housing defining a plurality of ridges extending outwardly therefrom and transverse to a longitudinal axis of the hub;
an absorbent member having a conically tapered outer surface configured to absorb a liquid and an inner surface defining a cavity; and
a nut movably disposed over the hub housing and being at least partially disposed within the cavity of the absorbent member, the nut having at least one finger sequentially engageable with the plurality of ridges for incrementally moving and securing the washer and absorbent member at any one of a plurality of positions along the longitudinal axis of the housing.

14. A nasal catheter assembly, comprising:
a balloon catheter having a first catheter defining a lumen, a second catheter disposed over the first catheter, and first and second balloons disposed over the first and second catheters, the first and second catheters each having a plurality of transverse openings extending transverse to respective longitudinal axes thereof and arranged relative to each other such that fluid flowing from the lumen and from the transverse openings fills and expands the first balloon and then the second balloon;
a hub connected to the balloon catheter and defining an inlet port in communication with the lumen of the balloon catheter such that fluid introduced through the inlet port is directed into the lumen; and
an absorbent member slidably disposed over the hub.

15. The assembly of claim 14, wherein the absorbent member is a conical sponge.

16. The assembly of claim 15, further comprising a washer disposed within the conical sponge, the washer being slidably disposed over the hub and rotationally constrained relative thereto.

17. The assembly of claim 14, wherein the first balloon extends over an end of the first catheter, and the lumen of the first catheter extends through the end.

18. The assembly of claim 14, wherein the second catheter has an outer surface defining a plurality of longitudinally extending ribs, and an inner surface defining a plurality of longitudinally extending ribs, the ribs of the inner surface engaging the first catheter, and the ribs of the outer surface interfacing with the first and second balloons.

19. The assembly of claim 14, wherein at least one pair of transverse openings of the first and second catheters feeding fluid to the first balloon are aligned, and each of the transverse openings of the first and second catheters feeding fluid to the second balloon are non-aligned.

20. The assembly of claim 14, wherein the hub further includes a first connector member and a second connector member connected to the first connector member, the first connector member defining the inlet port and including a one-way valve therein, the second connector member defining a connector end connected to the first end of the balloon catheter.
